(19) United States
(12) Patent Application Publication
(10) Pub. No.: US 2017/0100196 A1
(43) Pub. Date: Apr. 13, 2017

(54) SHAPE ESTIMATION DEVICE, ENDOSCOPE SYSTEM INCLUDING SHAPE ESTIMATION DEVICE, SHAPE ESTIMATION METHOD, AND PROGRAM FOR SHAPE ESTIMATION

(71) Applicant: OLYMPUS CORPORATION, Tokyo (JP)

(72) Inventors: Koichi Takayama, Koganei-shi (JP); Hiromasa Fujita, Hachioji-shi (JP); Ken Sato, Hachioji-shi (JP)

(73) Assignee: OLYMPUS CORPORATION, Tokyo (JP)

(21) Appl. No.: 15/384,791

(22) Filed: Dec. 20, 2016

Related U.S. Application Data
(63) Continuation of application No. PCT/JP2015/064959, filed on May 25, 2015.

(30) Foreign Application Priority Data
Jun. 26, 2014 (JP) 2014-131772

(51) Int. Cl. A61B 34/20; A61B 1/06; A61B 1/07; A61B 1/00

(52) U.S. Cl. CPC A61B 34/20; A61B 1/00045; A61B 1/0669; A61B 1/07; A61B 1/00004; A61B 2034/2061

(57) ABSTRACT

A shape estimation device includes an input circuit, a storage circuit and an arithmetic circuit. The input circuit receives light amount information being a relationship between a wavelength and a light amount. The light amount information is acquired by using a sensor configured such that the light amount to be detected with respect to the wavelength corresponding to each of sensing parts varies in accordance with a shape of each sensing part. The storage circuit stores a light amount estimation relationship including a relationship among the shape, the wavelength and the light amount. The arithmetic circuit calculates a light amount estimation value by an optimization arithmetic operation such that the estimation value based on the light amount estimation relationship and the light amount information satisfy a predetermined condition.

### CROSS-REFERENCE TO RELATED APPLICATIONS

This application is a Continuation Application of PCT Application No. PCT/JP2015/064959, filed May 25, 2015 and based upon and claiming the benefit of priority from prior the Japanese Patent Application No. 2014-131772, filed Jun. 26, 2014, the entire contents of all of which are incorporated herein by references.

### BACKGROUND OF THE INVENTION

1. Field of the Invention

The present invention relates to a shape estimation device, an endoscope system including the shape estimation device, a shape estimation method, and a program for shape estimation.

2. Description of the Related Art

There is known a device for detecting the shape of an endoscope. For example, Jpn. Pat. Appln. KOKAI Publication No. 2007-143600 discloses a technique relating to a shape detection probe using an optical fiber. The shape detection probe includes an optical fiber which bends as one piece with a scope of the endoscope. The optical fiber is provided with an optical modulator which modulates intensities, etc. of wavelength components being different from each other. KOKAI Publication No. 2007-143600 discloses that the shape detection probe can detect the shape of the endoscope, based on the intensities, etc. of wavelength components before and after modulation by the optical modulator. However, KOKAI Publication No. 2007-143600 fails to concretely disclose how to derive the shape of the endoscope, based on the intensities, etc. of the wavelength components.

### BRIEF SUMMARY OF THE INVENTION

One embodiment of the present invention is a shape estimation device comprising an input circuit configured to receive light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts, a storage circuit configured to store a light amount estimation relationship including shape characteristic information representing a relationship among the shape, the wavelength and the light amount with respect to each of the plurality of sensing parts, and a shape arithmetic circuit configured to calculate shape information being an estimation value of the shape by calculating a light amount estimation value by an optimization arithmetic operation such that the light amount estimation value calculated based on the light amount estimation relationship and the light amount information satisfy a predetermined condition, the light amount estimation value which is a relationship between the wavelength and the light amount.

Other embodiment of the present invention is an endoscope system comprising the above-described shape estimation device, an endoscope configured such that a light guide is provided in an insertion section, and an endoscope shape calculator configured to calculate a shape of the insertion section, based on the shape information.

Other embodiment of the present invention is a shape estimation method comprising acquiring light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts, acquiring a light amount estimation relationship including shape characteristic information representing a relationship among the shape, the wavelength and the light amount with respect to each of the plurality of sensing parts, and calculating shape information being an estimation value of the shape, by calculating a light amount estimation value by an optimization arithmetic operation such that the light amount estimation value calculated based on the light amount estimation relationship and the light amount information satisfy a predetermined condition, the light amount estimation value which is a relationship between the wavelength and the light amount.

Other embodiment of the present invention is a program for shape estimation, which causes a computer to execute acquiring light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts, acquiring a light amount estimation relationship including shape characteristic information representing a relationship among the shape, the wavelength and the light amount with respect to each of the plurality of sensing parts, and calculating shape information being an estimation value of the shape, by calculating a light amount estimation value by an optimization arithmetic operation such that the light amount estimation value calculated based on the light amount estimation relationship and the light amount information satisfy a predetermined condition, the light amount estimation value which is a relationship between the wavelength and the light amount.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1 is a block diagram which schematically shows a configurational example of an endoscope system according to an embodiment of the present invention.

FIG. 2 is a view which schematically shows a configurational example of a sensor driver and a sensor unit according to the embodiment.

FIG. 3 is a view showing an example of the relationship between the wavelength and intensity of light which is emitted by a light source.

FIG. 4 is a view showing an example of the relationship between the wavelength of light, which falls on a photodetector, and the detection sensitivity of the photodetector.

FIG. 5 is a cross-sectional view which schematically shows a configurational example of a sensing part.

FIG. 6 is a view showing an example of the relationship between the wavelengths of light and absorptivities in light absorbers.

FIG. 7A is a view for explaining the sensing part.

FIG. 7B is a view for explaining the sensing part.

FIG. 7C is a view for explaining the sensing part.

FIG. 8 is a view showing an example of the relationship between wavelength and a reference light amount.

FIG. 9 is a view showing an example of curvature characteristic information being the relationship between wavelength and a variation ratio in light amount in a first sensing part.

FIG. 10 is a view showing an example of curvature characteristic information being the relationship between wavelength and a variation ratio in light amount in a second sensing part.

FIG. 11 is a view showing an example of curvature characteristic information being the relationship between the curvatures and variation ratios in light amount of the first sensing part and second sensing part.

FIG. 12 is a view showing an example of the relationship between wavelength and a light amount, which is obtained by the photodetector.

FIG. 13 is a view showing an example of the relationship between wavelength, and a product between a variation ratio in light amount in the first sensing part and a variation ratio in light amount in the second sensing part.

FIG. 14 is a view showing an example of the relationship between wavelength, a variation ratio in light amount in the first sensing part and a variation ratio in light amount in the second sensing part.

FIG. 15 is a block diagram which schematically shows a configurational example of a shape optimization circuit according to a first calculation method.

FIG. 16 is a flowchart showing an example of an operation according to the first calculation method.

FIG. 17 is a flowchart showing an example of a shape information calculation process according to the first calculation method.

FIG. 18 is a block diagram which schematically shows a configurational example of a shape optimization circuit according to a second calculation method.

FIG. 19 is a flowchart showing an example of an operation according to the second calculation method.

FIG. 20 is a flowchart showing an example of a first shape information calculation process according to the second calculation method.

FIG. 21 is a flowchart showing an example of a second shape information calculation process according to the second calculation method.

FIG. 22 is a block diagram which schematically shows a configurational example of a shape optimization circuit according to a third calculation method.

FIG. 23 is a flowchart showing an example of a shape information calculation process according to the third calculation method.

### DETAILED DESCRIPTION OF THE INVENTION

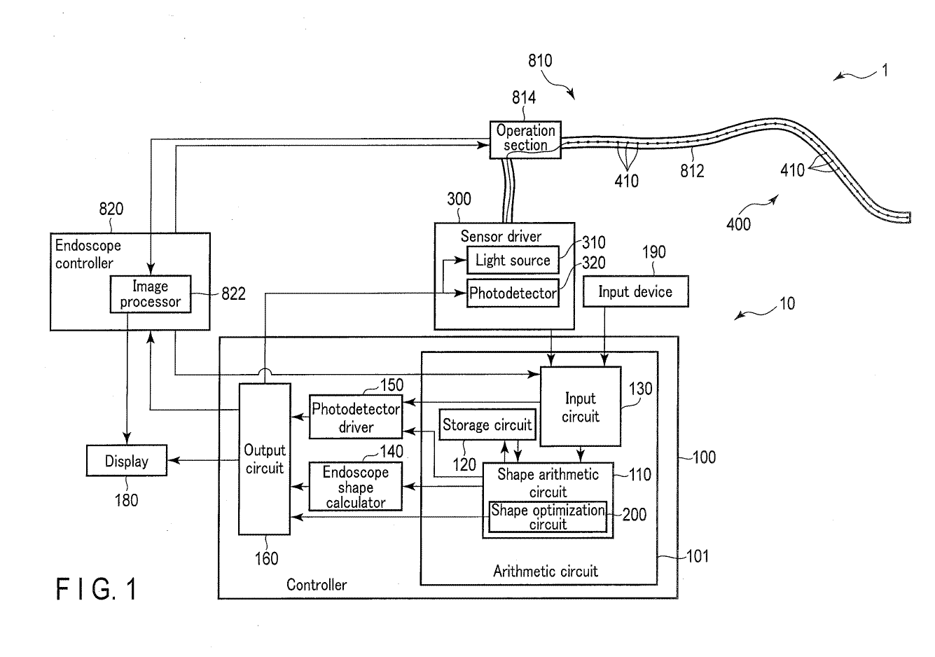

An embodiment of the present invention will be described with reference to the drawings. FIG. 1 schematically shows a configurational example of an endoscope system 1 in which a shape estimation device 10 according to the invention is used. As shown in FIG. 1, the endoscope system 1 includes an endoscope 810. The endoscope 810 includes an insertion section 812 which has an elongated shape and is free to bend, and an operation section 814 for performing various operations of the endoscope 810. The endoscope system 1 further includes an endoscope controller 820 for controlling various operations of the endoscope 810. The endoscope controller 820 is provided with an image processor 822 for processing an image acquired by the endoscope 810.

The endoscope system 1 includes the shape estimation device 10 according to the present invention. The shape estimation device 10 is a device which estimates a shape of the insertion section 812 in the endoscope 810. The endoscope system 1 further includes a display 180 and an input device 190. The display 180 is a general display device, and is, for instance, a liquid crystal display, a CRT display, or an organic EL display. The display 180 is connected to the endoscope controller 820, and displays an image acquired by the endoscope 810. In addition, the display 180 is connected to a controller 100 (to be described later) of the shape estimation device 10, and displays information of the shape of the insertion section 812 of the endoscope 810 acquired by the shape estimation device 10.

The input device 190 is a general device for input, and is, for instance, a keyboard, a mouse, a pointing device, a tag reader, a button switch, a slider, or a dial. The input device 190 is connected to the controller 100 (to be described later) of the shape estimation device 10. The input device 190 is used in order for a user to input various instructions for operating the shape estimation device 10. In addition, the input device 190 may be a storage medium. In this case, the information stored in the storage medium is input to the controller 100.

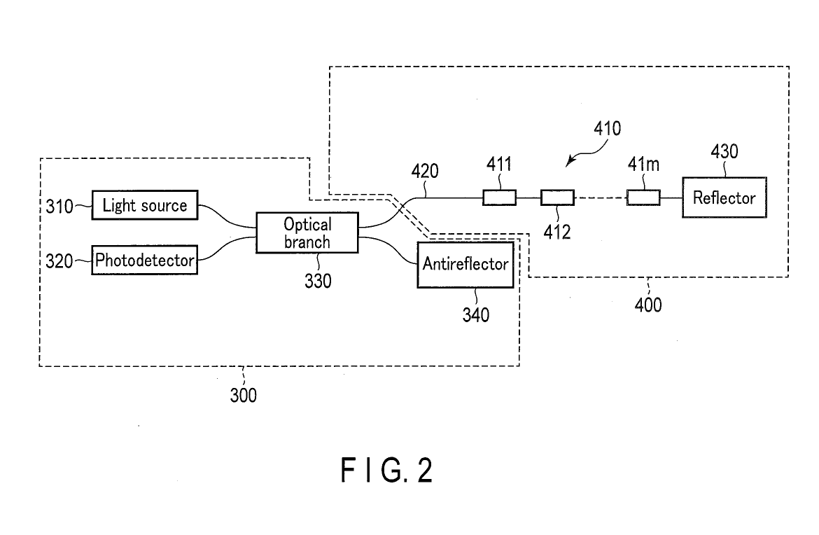

The shape estimation device 10 will be described. The shape estimation device 10 includes the controller 100, a sensor driver 300 and a sensor unit 400. The sensor driver 300 in the shape estimation device 10 includes a light source 310 and a photodetector 320. The sensor unit 400 includes a plurality of sensing parts 410 provided within the insertion section 812 in the endoscope 810. The outline of a configurational example of the sensor driver 300 and sensor unit 400 will be described with reference to FIG. 2.

As shown in FIG. 2, the sensor driver 300 includes the light source 310, the photodetector 320, an optical branch 330, and an antireflector 340.

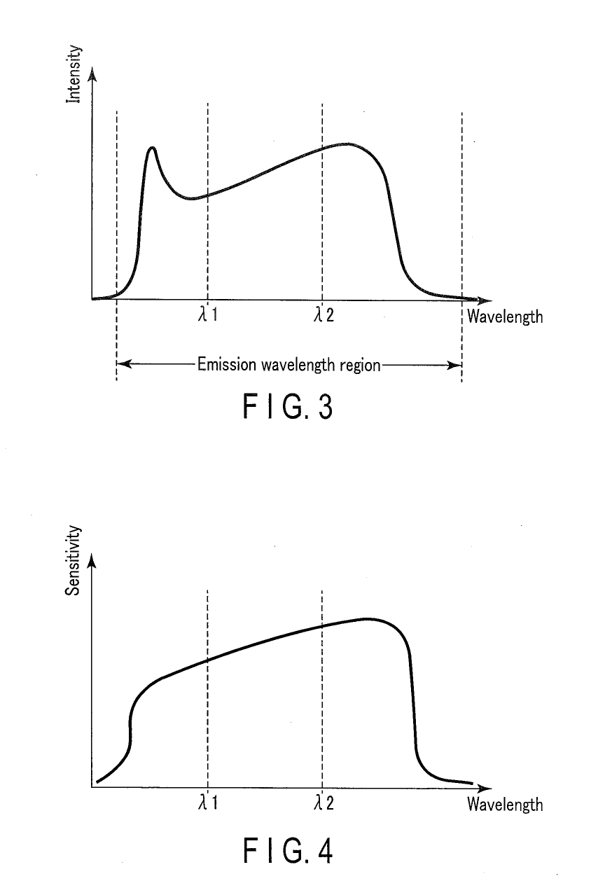

The light source 310 is, for example, a generally known light emission unit, such as a lamp, an LED, or a laser diode. The light source 310 may further include a fluorescent element for converting wavelength. The light source 310 emits light in a predetermined emission wavelength region. FIG. 3 is a view showing an example of the relationship between the wavelength and intensity of light which is emitted by a light source 310. The first wavelength λ1 as shown in FIG. 3 is a characteristic wavelength of a spectrum which a first sensing part 411 (to be described later) of the sensor unit 400 absorbs. Here, the characteristic wavelength is, for example, a wavelength at which absorption becomes maximum. Similarly, the second wavelength λ2 as shown in FIG. 3 is a characteristic wavelength of a spectrum which a second sensing part 412 of the sensor unit 400 absorbs. As shown in FIG. 3, the emission wavelength region includes wavelength to be used by the sensor unit 400, for example, the first wavelength λ1 or the second wavelength λ2.

The photodetector 320 includes an element for separation of light into its spectral components, such as a spectroscope or a color filter; and a light reception element such as a photodiode. The photodetector 320 detects the intensity of light in a predetermined wavelength region, and outputs light amount information. Here, the light amount information is information indicative of the relationship between a specific wavelength in the predetermined wavelength region and the intensity of light at this wavelength. FIG. 4 is a view showing an example of the relationship between the wavelength of light, which falls on the photodetector 320, and the detection sensitivity of the photodetector 320. As shown in FIG. 4, the photodetector 320 has detection sensitivity within a wavelength region including the above-described first wavelength λ1 and second wavelength λ2.

The photodetector 320 may be configured to acquire light amounts of various wavelengths at the same time, or may be configured to acquire these light amounts in a time-division manner. In addition, the light source 310 may be configured to successively emit lights of different wavelengths in a time-division manner, and the photodetector 320 may not include the element for separation of light into its spectral components and may be configured to detect light amounts of different wavelengths in a time-division manner.

The optical branch 330 includes an optical coupler or a semitransparent mirror. The optical branch 330 guides light emitted from the light source 310 to a light guide 420 (to be described later) of the sensor unit 400, and guides light guided by the light guide 420 to the photodetector 320.

The antireflector 340 is a member which absorbs that part of the light emitted from the light source 310, which did not enter the light guide 420. The antireflector 340 functions to prevent a part of the light emitted from the light source 310, which did not enter the light guide 420, from returning to the photodetector 320.

The sensor unit 400 includes the plurality of sensing parts 410, the light guide 420 and a reflector 430. The light guide 420 is, for example, an optical fiber, and has flexibility. The light guide 420 is provided within the insertion section 812 of the endoscope 810 along the longitudinal direction of the insertion section 812. The light guide 420 is arranged on a region where the shape is to be calculated in the insertion section 812. As described above, the light emitted from the light source 310 enters the light guide 420 via the optical branch 330. The light guide 420 guides the fell light along the light guide 420.

The light guide 420 is provided with the plurality of sensing parts 410. The sensing parts 410 include a first sensing part 411 and a second sensing part 412. Similarly, the sensing parts 410 include an m-th sensing part 41m. Here, “m” is an arbitrary number. The first sensing part 411, the second sensing part 412, etc. are arranged at different positions in the longitudinal direction of the light guide 420.

The outline of a configurational example of the sensing part 410 will be described with reference to FIG. 5. FIG. 5 is a view which schematically shows a cross section perpendicular to the longitudinal axis of the light guide 420. The sensing part 410 has such a configuration that a light absorber 429 is coated on an exposed part of a core 423 exposed by removing parts of a coating 421 and a cladding 422 of the light guide 420 that is, for example, an optical fiber. Light absorbers 429 having different light absorptivities for respective wavelengths are used for the respective sensing parts 410.

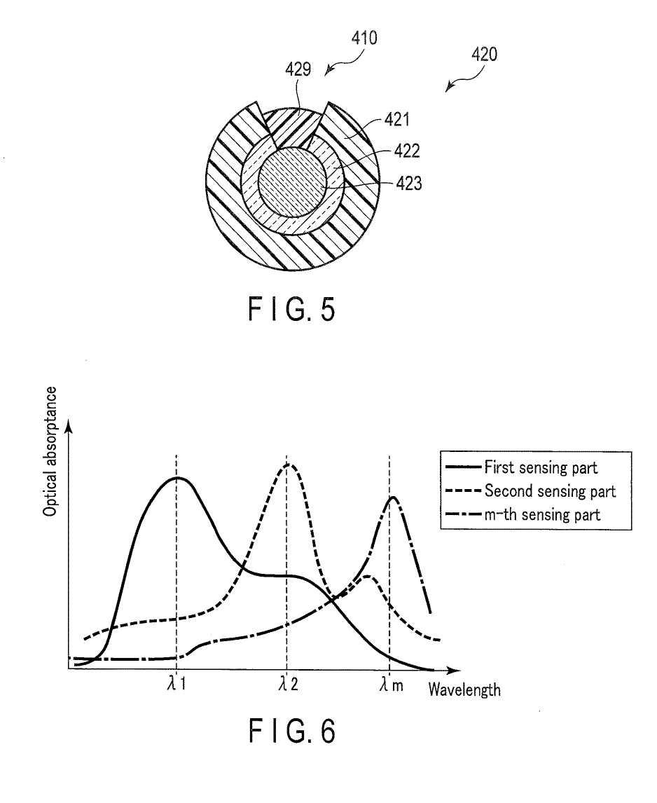

FIG. 6 shows an example of the relationship between the wavelengths of light and absorptivities in the light absorbers 429. In FIG. 6, a solid line indicates absorption characteristics of the light absorber 429 provided in the first sensing part 411, a broken line indicates absorption characteristics of the light absorber 429 provided in the second sensing part 412, and a dot-and-dash line indicates absorption characteristics of the light absorber 429 provided in the m-th sensing part 41m. As shown in FIG. 6, the light absorbers 429 provided in the different sensing parts 410 have absorption characteristics being different from each other. The wavelength with the highest light absorptivity in the light absorber 429 provided in the first sensing part 411 is referred to as the above-described first wavelength λ1, the wavelength with the highest light absorptivity in the light absorber 429 provided in the second sensing part 412 is referred to as the above-described second wavelength λ2, and the wavelength with the highest light absorptivity in the light absorber 429 provided in the m-th sensing part 41m is referred to as an m-th wavelength λm.

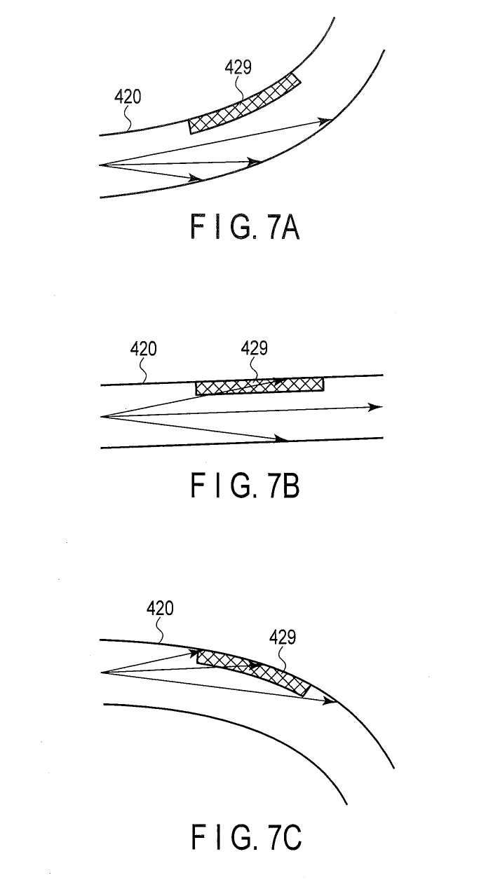

The sensing part 410 will be further described with reference to FIG. 7A, FIG. 7B and FIG. 70. FIG. 7A, FIG. 7B and FIG. 7C are views which schematically show the shapes of the light guide 420 and the optical path in the light guide 420 at the time of each shape. For example, as shown in FIG. 7A, when the light guide 420 bends such that the light absorber 429 is located inside, the light fell on the light absorber 429 is relatively small, and the optical transmissibility by the light guide 420 increases. On the other hand, as shown in FIG. 7C, when the light guide 420 bends such that the light absorber 429 is located outside, the light fell on the light absorber 429 is relatively large, and the optical transmissibility by the light guide 420 decreases. As shown in FIG. 7B, when the light guide 420 is not bent, the optical transmissibility by the light guide 420 is lower than in the case shown in FIG. 7A, and is higher than in the case shown in FIG. 7C. For the purpose of the description below, it is assumed that the bend of the light guide 420 in such a direction that the optical transmissibility by the light guide 420 increases, as shown in FIG. 7A, is referred to as the bend in a positive direction, and that the bend of the light guide 420 in such a direction that the optical transmissibility by the light guide 420 decreases, as shown in FIG. 7C, is the bend in a negative direction. In this manner, for example, the respective light absorbers 429 function as optical members which exert different effects on the spectrum of light guided by the light guide.

Referring back to FIG. 2, the sensor driver 300 and the sensor unit 400 will be described. The reflector 430 is provided at an end portion of the light guide 420, that is, a distal end thereof, on the side on which the light guide 420 is not connected to the optical branch 330. The reflector 430 reflects the light guided from the optical branch 330 by the light guide 420 in a direction of the optical branch 330.

By the above configuration, the light emitted from the light source 310 is guided to the light guide 420 via the optical branch 330. This light is transmitted in the light guide 420 from the proximal-end side to the distal-end side. At this time, in the sensing part 410 provided on the light guide 420, the light is absorbed in accordance with the bend state of this sensing part 410, and the amount of transmitted light decreases at each wavelength. This light is reflected by the reflector 430, and the reflected light is transmitted in the light guide 420 from the distal-end side to the proximal-end side. This reflected light is guided to the photodetector 320 via the optical branch 330. The photodetector 320 detects the intensity of the guided light at each wavelength. Based on the detected intensity at each wavelength detected by the photodetector 320, the bend state of each sensing part 410, that is, the shape of the insertion section 812 of the endoscope 810 can be derived.

The sensor driver 300 including the optical branch 330, and the sensor unit 400 may be fixed or may be configured to be detachably attached.

The controller 100 will be described. The controller 100 is composed of an electronic calculator which is, for instance, a personal computer. The controller 100 includes an arithmetic circuit 101, an endoscope shape calculator 140, a photodetector driver 150, and an output circuit 160.

The arithmetic circuit 101 is composed of, for example, a device including a CPU or an ASIC. The arithmetic circuit 101 includes a shape arithmetic circuit 110, a storage circuit 120 and an input circuit 130.

Light amount information relating to the wavelength and detected light intensity is input to the input circuit 130 from the photodetector 320. The input circuit 130 transmits the input light amount information to the shape arithmetic circuit 110. In addition, a signal or the like representing a user's instruction that was input by the input device 190 is input to the input circuit 130. Besides, for example, identification information for identifying the type or the individual of the sensor unit 400 is input to the input circuit 130. In addition, shape characteristic information of the sensing parts 410 may be input to the input circuit 130. Further, information output from the endoscope controller 820 may be input to the input circuit 130. The input circuit 130 transmits the input signals to the photodetector driver 150 or shape arithmetic circuit 110.

The storage circuit 120 stores various kinds of information being necessary for arithmetic operations that are executed by the shape arithmetic circuit 110. The storage circuit 120 stores, for example, programs including a calculation algorithm, and a light amount estimation relationship including shape characteristic information (to be described later) of the sensing parts 410.

The shape arithmetic circuit 110 calculates the shape of each sensing part 410, based on the light amount information acquired via the input circuit 130, and the information stored in the storage circuit 120. The shape arithmetic circuit 110 includes a shape optimization circuit 200 to be used for shape calculation. The details of the shape optimization circuit 200 will be described later. The shape arithmetic circuit 110 transmits the calculated shape of the sensing part 410 to the endoscope shape calculator 140 and output circuit 160. In addition, the shape arithmetic circuit 110 outputs to the photodetector driver 150 the information relating to the operation of the photodetector 320 being necessary for the shape calculation, such as a gain of the photodetector 320.

The endoscope shape calculator 140 including the controller includes, for example, a CPU or an ASIC. Based on the shape of each sensing part 410 calculated by the shape arithmetic circuit 110, the endoscope shape calculator 140 calculates the shape of the insertion section 812 of the endoscope 810, in which the sensing parts 410 are arranged. The calculated shape of the insertion section 812 is transmitted to the output circuit 160. The endoscope shape calculator 140 may be assembled in the shape arithmetic circuit 110.

The photodetector driver 150 generates a driving signal of the photodetector 320, based on the information acquired from the input circuit 130. By this driving signal, the photodetector driver 150 switches on/off the operation of the photodetector 320, for example, based on the user's instruction input to the input device 190 and is acquired via the input circuit 130, or adjusts the gain of the photodetector 320, based on the information acquired from the shape arithmetic circuit 110. In addition, the photodetector driver 150 may be configured to also control the operation of the light source 310. The photodetector driver 150 transmits the generated driving signal to the output circuit 160.

The output circuit 160 outputs to the display 180 the shape of the insertion section 812 acquired from the endoscope shape calculator 140 to the display 180, and causes the display 180 to display the shape of the insertion section 812. In addition, the output circuit 160 outputs to the endoscope controller 820 the shape of the sensing part 410 acquired from the shape arithmetic circuit 110. The endoscope controller 820 controls the operation of the endoscope 810, based on the acquired shape of the insertion section 812 and the acquired shape of the sensing part 410. Besides, the output circuit 160 outputs the driving signal acquired from the photodetector driver 150 to the photodetector 320, and drives the photodetector 320.

Next, the operation of the endoscope system 1 according to the present embodiment will be described. The insertion section 812 of the endoscope 810 is inserted in an insertion target by the user. During insertion, the insertion section 812 bends in accordance with the shape of the insertion target. The endoscope 810 performs image by an image device provided in the distal end of the insertion section 812. The image signal acquired by the image is transmitted to the image processor 822 of the endoscope controller 820. The image processor 822 creates an endoscopic image, based on the acquired image signal. The image processor 822 causes the display 180 to display the created endoscopic image.

When the user wishes to cause the display 180 to display the shape of the insertion section 812, or when the user wishes to cause the endoscope controller 820 to perform various operations using the shape of the insertion section 812, the user inputs the corresponding instruction to the controller 100 through the input device 190. Then, the shape estimation device 10 operates.

If the shape estimation device 10 operates, the light source 310 of the sensor driver 300 emits light of a predetermined emission wavelength region. The light emitted from the light source 310 is guided to the light guide 420 of the sensor unit 400 via the optical branch 330. The guided light is reflected on the reflector 430 provided on the distal end of the light guide 420, and propagated in the light guide 420 from the proximal-end side to the distal-end side. When the light travels in the light guide 420, light amount of each wavelength varies in accordance with the shape of the sensing parts 410. The light reaches the photodetector 320 via the optical branch 330.

The photodetector 320 detects the intensity of the light, which has reached at the photodetector 320, at each wavelength. The photodetector 320 outputs the detected light intensity to the input circuit 130 of the controller 100. The shape arithmetic circuit 110 calculates the shape of each sensing part 410, based on the light intensity detected by the photodetector 320 and acquired via the input circuit 130.

The endoscope controller 820 acquires information of the shape of each sensing part 410 from the shape arithmetic circuit 110 via the output circuit 160. The endoscope controller 820 controls the operation of the endoscope 810 by using the information of the shape of each sensing part 410.

In addition, the endoscope shape calculator 140 acquires information of the shape of each sensing part 410 from the shape arithmetic circuit 110. Based on the shape of each sensing part 41, the endoscope shape calculator 140 calculates the shape of the insertion section 812 of the endoscope 810. The endoscope shape calculator 140 causes, via the output circuit 160, the display 180 to display the calculated shape of the insertion section 812. Furthermore, the endoscope controller 820 acquires information of the shape of the insertion section 812 from the endoscope shape calculator 140 via the output circuit 160, and uses the information for the control of the endoscope 810.

In this manner, according to the shape estimation device 10, the shape of each sensing part 410 is acquired. Based on the acquired shape of the sensing part 410, the endoscope shape calculator 140 calculates the shape of the insertion section 812 of the endoscope 810. Thereby, the user can understand the shape of the insertion section 812 while operating the endoscope 810. In addition, the endoscope controller 820 performs various arithmetic calculation based on the shape of the sensing parts 410. Thereby, the endoscope controller 820 can properly control the operation of the endoscope 810 in accordance with the shape of the insertion section 812.

Next, a detailed description will be given of arithmetic operations which are executed by the arithmetic circuit 101 in the shape estimation device 10 of the present embodiment. For easier understanding, a case in which only the first sensing part 411 and second sensing part 412 are included in the sensing parts 410 is described by way of example.

To begin with, information to be prepared in advance before the use of the endoscope system 1, will be described. A light amount D_{λn} of light of wavelength λn, which is detected by the photodetector 320, is given by the following equation (1).

D_{λn}=E_{λn}×A_{λn}×B_{λn}×L_{λn} equation (1)

Here, E_{λn} is a light amount of light of wavelength λn, which is emitted from the light source 310; A_{λn} is an absorptivity of light of wavelength An in the first sensing part 411; B_{λn} is an absorptivity of light of wavelength λn in the second sensing part 412; and L_{λn} is an absorptivity of light of wavelength λn by members other than the sensing parts 410, such as the optical branch 330, light guide 420 and reflector 430.

The emission light amount E_{λn} and absorptivity L_{λm} do not depend on the shape of the sensing part 4′10. Accordingly, the light amount D_{λn} can be rewritten as hereinafter. Specifically, the light amount of light of wavelength λn, which is detected by the photodetector 320 when each sensing part 410 is in a predetermined referenced shape (hereinafter referred to as “reference shape”), is calculated in advance as a reference light amount I_{λn}. In addition, the ratio between the light amount of light of wavelength λn, which is detected by the photodetector 320 when all sensing parts 410 (in this example, the second sensing part 412), other than the first sensing part 411, are in the referenced shape, and the reference light amount I_{λn}, is set as a variation ratio α_{λn} in the first sensing part 411. Besides, the ratio between the light amount of light of wavelength λₙ, which is detected by the photodetector 320 when all sensing parts 410 (in this example, the first sensing part 411), other than the second sensing part 412, are in the referenced shape, and the reference light amount I_{λn}, is set as a variation ratio β_{λn} in the second sensing part 412. At this time, the light amount D_{λn} is given by the following equation (2).

D_{λn}=I_{λn}×α_{λn}×β_{λn} equation (2)

The light absorptivity in each sensing part 410 varies in accordance with the shape of each sensing part 410, for example, curvature κ. Accordingly, the above-described ratio α_{λn} in the first sensing part 411 is given by the following equation (3).

α_{λn}=f_{λn}(κ_{α}) equation (3)

Here, κ_{α} is the curvature of the first sensing part 411, and a function f_{λn} is a variation ratio which is stored in the storage circuit 120 as a curvature characteristic information.

Similarly, the above-described ratio β_{λn} is given by the following equation (4).

β_{λn}=g_{λn}(κ_{β}) equation (4)

Here, κ_{β} is the curvature of the second sensing part 412, and a function g_{λn} is a variation ratio which is stored in the storage circuit 120 as a curvature characteristic information.

From equation (2), equation (3) and equation (4), the following equation (5) is obtained.

D_{λn}(κ_{α},κ_{β})=I_{λn}×f_{λn}(κ_{α})×g_{λn}(κ_{β}) equation (5)

For the reference shape for determining the reference light amount I_{λn}, for example, the case is adopted in which all of the sensing parts 410 are in the straight shape, that is, the case in which the curvature is 0 and the radius of curvature thereof is ∞. However, the reference shape is not limited to this case, and the reference shape may be a shape other than the straight shape. In addition, the reference shape may not be the same shape with respect to all sensing parts, and arbitrary shapes may be set for the respective sensing parts.

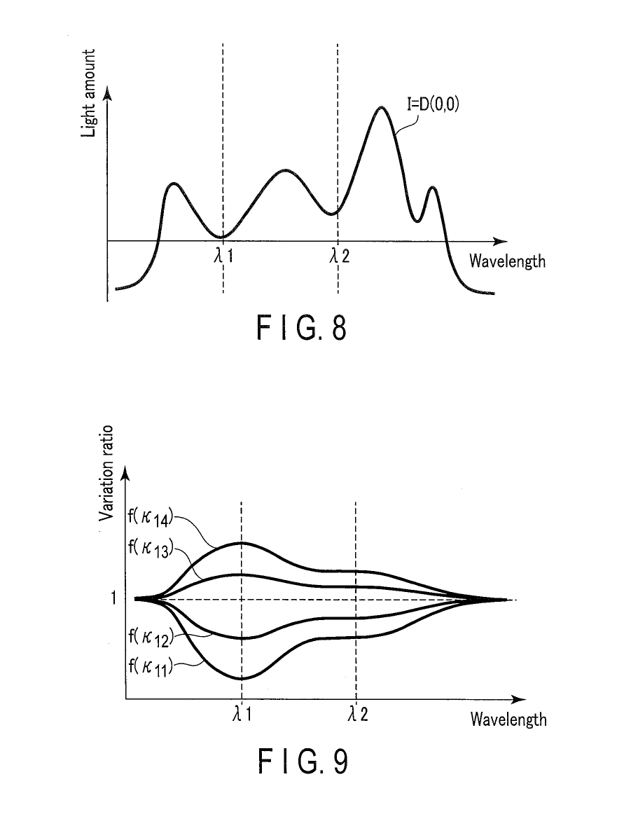

Hereinafter, a description will be given of the case in which the reference shape that is the straight shape is adopted for all sensing parts 410. The relationship between wavelength and a reference light amount I shows a spectrum such as FIG. 8. A light amount D_{λn}(0, 0) at a time when all sensing parts 410 are in the reference shape is, by definition, given by the following equation (6).

D_{λn}(0,0)=I_{λn} equation (6)

Specifically, by definition, the reference light amount is I_{λn}, f_{λn}(0)=1, and g_{λn}(0)=1.

The function f_{λn} and function g_{λn} being curvature characteristic information can be obtained by varying, in a state in which the shapes of the sensing parts, other than a target sensing part, are set to be the reference shape, the curvature of the target sensing part within a possible range.

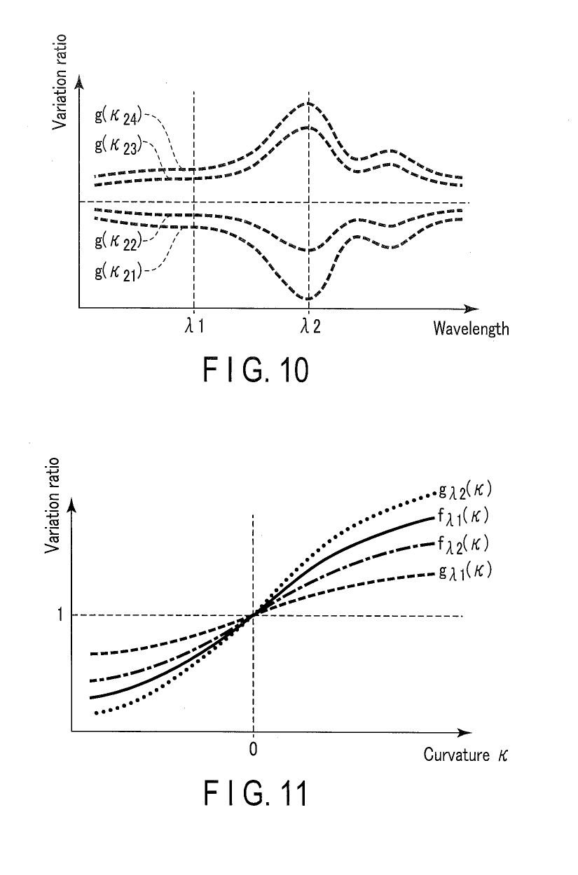

The curvature characteristic information of the first sensing part 411, that is, the relationship between the wavelength and variation ratio, is indicated by spectra as shown in, for example, FIG. 9. Here, the curvature κα is κ₁₁<κ₁₂<κ₁₃<κ₁₄. In addition, the curvature characteristic information of the second sensing part 412, that is, the relationship between the wavelength and variation ratio, is indicated by spectra as shown in, for example, FIG. 10. Here, the curvature κ_{β} is κ₂₁<κ₂₂<κ₂₃<κ₂₄.

Each of FIG. 9 and FIG. 10 shows curvature characteristic information relating to four curvatures. However, as the curvature characteristic information, the relationship between the wavelength and variation ratio in the emission wavelength range are obtained with respect to various curvatures as shown in FIG. 9 and FIG. 10. In this manner, the curvature characteristic information of the first sensing part 411, which represents the relationship between the curvature κ_{α} and variation ratio α_{λn}, and the curvature characteristic information of the second sensing part 412, which represents the relationship between the curvature κ_{β} and variation ratio β_{λn}, are obtained as shown in FIG. 11.

In the meantime, from equation (5), the function f_{λn}(κ_{α}) is given by the following equation (7).

$${f}_{λ n} ( {κ}_{α} ) = \frac{{D}_{λ n} ( {κ}_{α} , 0 )}{{I}_{λ n}} \qquad equation ( 7 )$$

Similarly, the function g_{λn}(κ_{β}) is given by the following equation (8).

$${g}_{λ n} ( {κ}_{β} ) = \frac{{D}_{λ n} ( 0 , {κ}_{β} )}{{I}_{λ n}} \qquad equation ( 8 )$$

The curvature characteristic information and reference light amount information I_{λn}, as shown in FIG. 8, are acquired in advance, for example, when the endoscope system 1 is manufactured, or when the endoscope system 1 is installed, and are prestored in the storage circuit 120. The curvature characteristic information and reference light amount I_{λn} may be acquired at each time of use. For example, at a time of starting the endoscope system 1, the reference light amount I_{λn} may be actually measured by setting the insertion section 812 in the straight shape, and then the shape of the insertion section 812 may be successively changed to predetermined bent shapes, and the curvature characteristic information may be acquired from the light amount detected at this time.

The curvature characteristic information and reference light amount I_{λn} may be stored not in the storage circuit 120 provided in the controller 100, but in a storage provided in the sensor driver 300 or sensor unit 400. By doing so, such characteristic information can be used even when the sensor driver 300 or sensor unit 400 is connected to other devices.

Additionally, for example, such a configuration may be adopted that the sensor driver 300 or sensor unit 400 is provided with identification information including a value inherent to the device, and the storage circuit 120 may store the curvature characteristic information and reference light amount I_{λn} associated with this identification information. By doing so, the controller 100 can manage and use the curvature characteristic information and reference light amounts I_{λn} of a plurality of sensor drivers 300 and sensor units 400. The identification information may be input from a keyboard that is the input device 190, may be read out from an RF-ID tag, or may be read out from the storage provided in the sensor driver 300 or sensor unit 400.

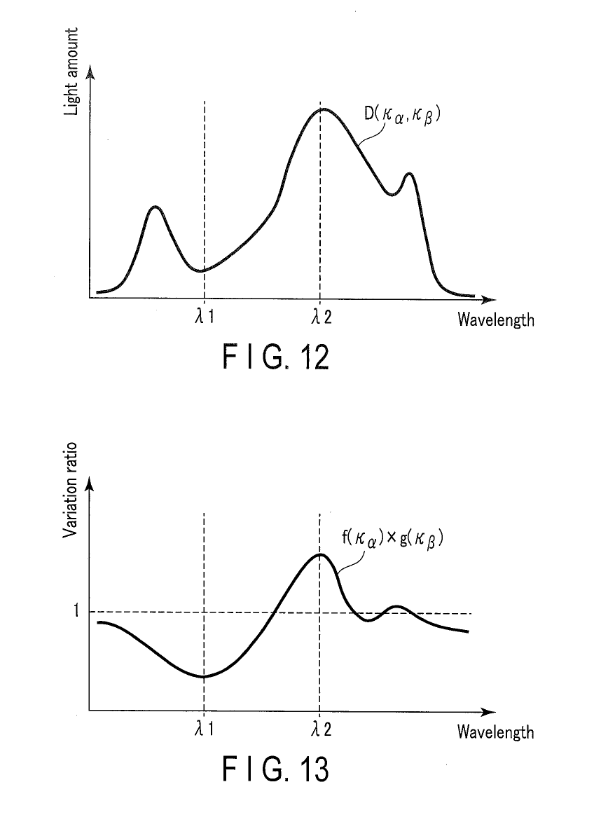

Next, arithmetic operations to be executed at the time of using the endoscope system 1 will be described. The case is now considered in which the curvatures of the first sensing part 411 and the second sensing part 412 are arbitrary curvatures κ_{α} and κ_{β}, respectively. Here, a light amount D (κ_{α}, κ_{β}) indicates a spectrum shown in, for example, FIG. 12. This light amount D (κ_{α}, κ_{β}) is acquired by the photodetector 320.

Based on equation (5) and the pre-acquired reference light amount I_{λn} as shown in FIG. 8, f(κ_{α})×g(κ_{β}) can be calculated, which is a product of variation ratio of the first sensing part 411 and the variation ratio of the second sensing part 412. The relationship between the wavelength and f(κ_{α})×g(κ_{β}) is, for example, as shown in FIG. 13.

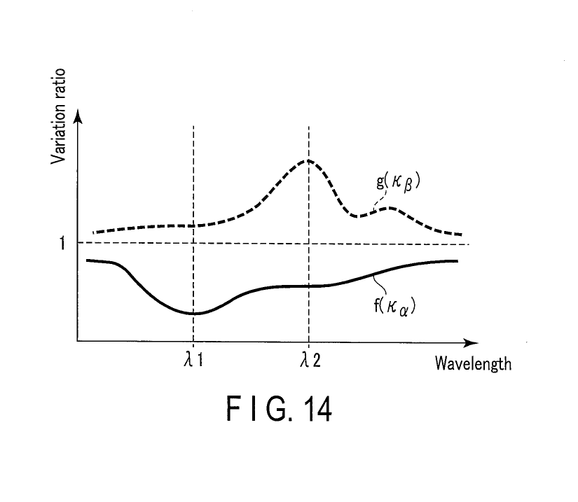

For example, if the relationship between the wavelength and f(κ_{α})×g(κ_{β}), which is shown in FIG. 13, can be separated into the relationship between the wavelength and f(K_{α}) and the relationship between the wavelength and g(κ_{β}), as shown in FIG. 14, the curvature κₐ of the first sensing part 411 and the curvature κ_{β} of the second sensing part 412 can be calculated from the light amount D (κ_{α}, κ_{β}).

As an example, in order to calculate the curvature κ_{α} of the first sensing part 411 and the curvature κ_{β} of the second sensing part 412, attention is now paid to the first wavelength λ1 and second wavelength λ2, and consideration is given to the following equation (9) with respect to the light amount D_{λ1} and light amount D_{λ2} at respective wavelengths detected by the photodetector 320.

$$\{ \begin{matrix}{D}_{λ 1} ( {κ}_{α} , {κ}_{β} ) = {I}_{λ1} \times {f}_{λ1} ( {κ}_{α} ) \times {g}_{λ1} ( {κ}_{β} ) \\ {D}_{λ 2} ( {κ}_{α} , {κ}_{β} ) = {I}_{λ2} \times {f}_{λ2} ( {κ}_{α} ) \times {g}_{λ2} ( {κ}_{β} )\end{matrix} \qquad equation ( 9 )$$

The storage circuit 120 prestores I_{λ1} and I_{λ2} being reference light amounts, and f_{λ1}, f_{λ2}, g_{λ1} and g_{λ2} being curvature characteristic information. Thus, based on the light amount D_{λ1} and light amount D_{λ2}, the curvature κ_{α} of the first sensing part 411 and the curvature κ_{β} of the second sensing part 412 can be calculated.

Although the case in which the sensing parts 410 includes two sensing parts, namely the first sensing part 411 and second sensing part 412, was described by way of example, the same calculations can be executed even when the number of sensing parts is three or more.

In the above example, in order to calculate the curvature κ_{α} of the first sensing part 411 and the curvature κ_{β} of the second sensing part 412, the first wavelength λ1 and second wavelength λ2 are used, but the restriction to this is unnecessary. Wavelengths other than the first wavelength λ1 and second wavelength Λ2 may be used. However, the precision in calculation of curvature is enhanced if use is made of the wavelength at which the light absorptivity of the light absorber 429 of each sensing part 410 becomes maximum.

Additionally, the wavelength used here may have a certain degree of band width. However, it is preferable that the band including the wavelength, at which the light absorptivity of the light absorber 429 becomes maximum, is used. By using the light amount for the wavelength having a bandwidth for arithmetic operations, it becomes unnecessary to greatly increase the resolution of the photodetector 320, and the reduction in cost of the shape estimation device 10 can be realized. Besides, since local wavelengths are not used, the robustness to the influence of noise is advantageously increased. A plurality of wavelength bands to be used for arithmetic operations may partly overlap. As regards the wavelengths to be used in arithmetic operations, the same applies to the following description.

Additionally, as regards the above equation (9), logarithms may be taken. By using logarithms, the calculation can be facilitated.

[First Calculation Method]

In the above description, the example was shown in which the number of sensing parts 410 is two, and the curvature κ_{α} of the first sensing part 411 and the curvature κ_{β} of the second sensing part 412 are calculated. However, in general, the number of sensing parts is arbitrary, and the case of two or more is considered here. In addition, the parameter representing the shape of the sensing part 410 is not limited to the curvature, and may include rotation, etc. In the description below, consideration will be given to shape information k1 to km being information representing the shapes of the sensing parts of the first sensing part 411 to the m-th sensing part 41m. The shape information may include various values representing shapes, such as curvature, a radius of curvature, an angle of bend, an angle of rotation, etc. The information referred to as “curvature characteristic information” in the above example, will be referred to as “shape characteristic information”.

Hereinafter, in order to calculate the shape information k1 to km, an example of executing a convergence calculation is described. Specifically, it is now considered that, when a light amount estimation value repeatedly calculated in the convergence calculation is set as E_{λx}, the shape information k1 to km is obtained by converging the light amount estimation value E_{λx} to a light amount D_{λx} detected by the photodetector 320.

For example, in the case of the sensing parts 410 including only the first sensing part 411 and second sensing part 412 described with reference to the above equation (5), the light amount estimation value E_{λx} corresponds to the following equation (10).

E_{λx}(κ₁,κ₂)=I_{λx}×f_{λx}(κ₁)×g_{λx}(κ₂) equation (10)

Here, κ₁ is an estimation value of the curvature of the first sensing part 411, and κ₂ is an estimation value of the curvature of the second sensing part 412. In this manner, the light amount estimation value E_{λx} is given as a function of the shape information k1 to km of the first to m-th sensing parts. For example, a light amount estimation equation for calculating the light amount estimation value corresponding to equation (10) is stored in the storage circuit 120.

Using the light amount D_{λx} detected by the photodetector 320 and the light amount estimation value E_{λx}, an evaluation value J is defined as in the following equation (11).

$$J = {\sum }_{x = 1}^{pl} {( {D}_{λ x} - {E}_{λ x} ( k 1 , … , km ) )}^{2} \qquad equation ( 11 )$$

Here, p is the number of wavelengths used for arithmetic operations, and p is m or more. Specifically, the evaluation value J is a value obtained by adding the square of an error of the light amount estimation value E_{λx} calculated by using the light amount estimation value, relative to the detected light amount D_{λx}.

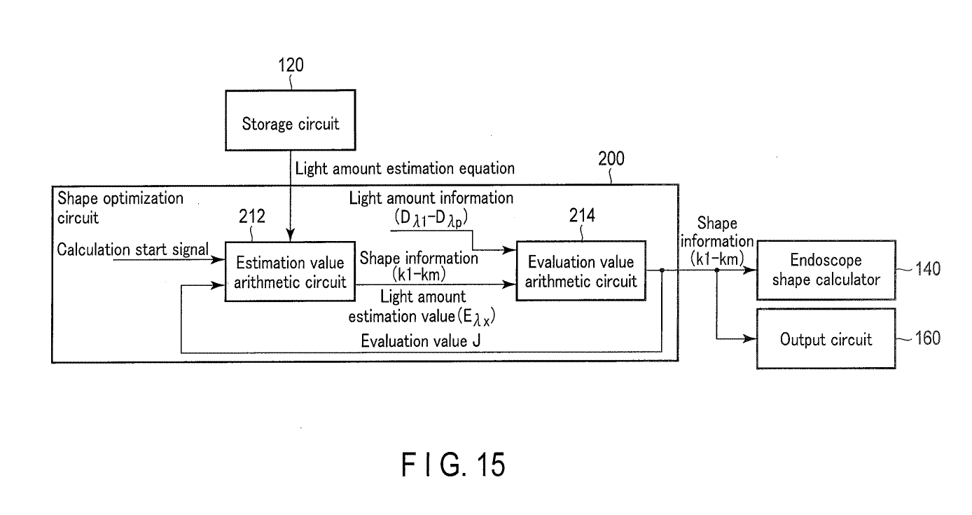

The convergence calculation is executed by the shape optimization circuit 200 in the shape arithmetic circuit 110. FIG. 15 shows the outline of a configurational example of the shape optimization circuit 200 according to the present calculation method. As shown in FIG. 15, the shape optimization circuit 200 includes an estimation value arithmetic circuit 212 and an evaluation value arithmetic circuit 214.

The estimation value arithmetic circuit 212 acquires the light amount estimation equation from the storage circuit 120. A calculation start signal is input to the estimation value arithmetic circuit 212. When the calculation start signal has been input, the estimation value arithmetic circuit 212 starts calculation of the light amount estimation value E_{λx}. The estimation value arithmetic circuit 212 outputs the calculated light amount estimation value E_{λx} and shape information k1 to km to the evaluation value arithmetic circuit 214.

The evaluation value arithmetic circuit 214 calculates the evaluation value J by using the equation (11), based on the light amount estimation value E_{λx} acquired from the estimation value arithmetic circuit 212 and the light amount D_{λx} acquired by the photodetector 320. If the evaluation value J is greater than a predetermined threshold A, the evaluation value arithmetic circuit 214 returns the evaluation value J to the estimation value arithmetic circuit 212, and causes the estimation value arithmetic circuit 212 to repeat the calculation of the light amount estimation value E_{λx}. On the other hand, if the evaluation value J is not greater than the predetermined threshold A, the evaluation value arithmetic circuit 214 outputs the shape information acquired from the estimation value arithmetic circuit 212 to the endoscope shape calculator 140 and output circuit 160.

The algorithm used in the present calculation method is a single optimization algorithm, or a composite optimization method in which some optimization algorithms are combined. The algorithm that is used may be any kind of algorithm. Examples of the algorithm that is used include population-based descent methods including Particle Swarm Optimization (PSO) and Differential Evolution (DE), Genetic Algorithm (GA), Simulated Annealing (SA), Newton's method, steepest descent method, least squares method, and simplex method.

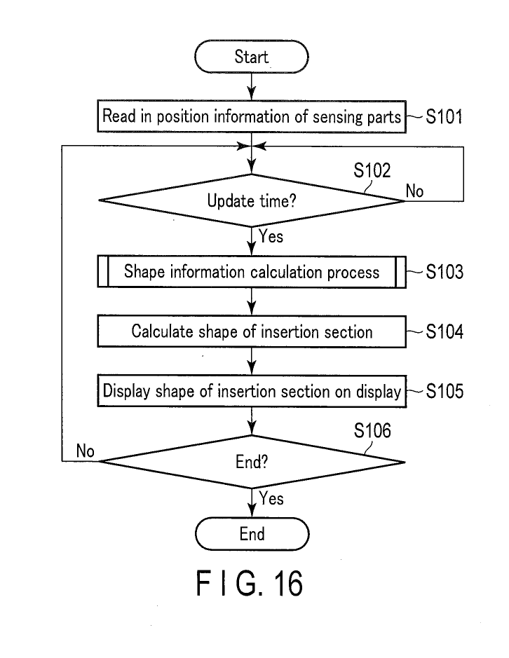

The operation of the controller 100 using the first calculation method will now be described with reference to a flowchart of FIG. 16. The operation to be described here is an operation of displaying on the display 180 the shape of the insertion section 812 of the endoscope 810. The operation is started, for example, when the user has requested, with use of the input device 190, that the shape of the insertion section 812 be displayed on the display 180.

In step S101, the controller 100 reads in position information of the sensing parts 410 stored in the storage circuit 120. Here, the position information is information relating to the position of each sensing part 410 relative to the insertion section 812 of the endoscope 810. The position information is used when the shape of the insertion section 812 is calculated based on the shapes of the sensing parts 410.

In step S102, the controller 100 determines whether a predetermined update time has come. Here, the update time means a time of updating the display of the shape of the insertion section 812 on the display 18. Specifically, in the present operation, the calculation and display of the shape of the insertion section 812 are repeated at every update time. In the determination of step S102, if it is determined that the update time has not come, the process repeats step S102, and stands by until the update time has come. On the other hand, if it is determined that the update time has come, the process advances to step S103.

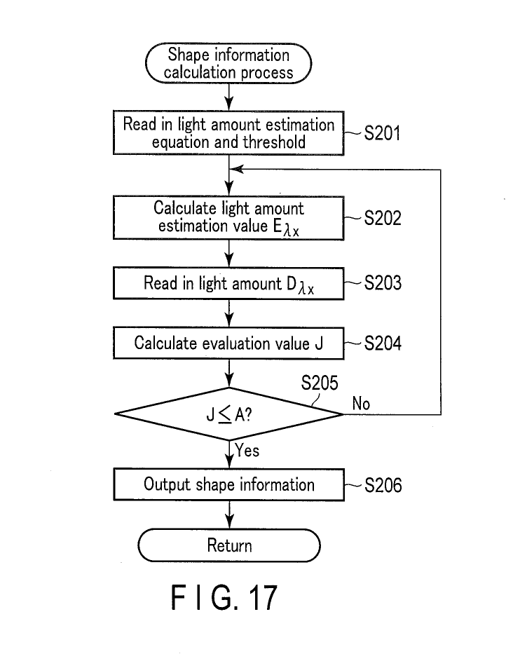

In step S103, the controller 100 executes a shape information calculation process according to the first calculation method. Specifically, the calculation start signal is input to the estimation value arithmetic circuit 212, and the shape information calculation process by the shape optimization circuit 200 is started. Referring to a flowchart of FIG. 17, the shape information calculation process will be described.

In step S201, the estimation value arithmetic circuit 212 of the shape optimization circuit 200 reads in the light amount estimation equation and predetermined threshold A from the storage circuit 120, the light amount estimation equation and predetermined threshold A being used in subsequent arithmetic operations.

In step S202, based on the light amount estimation equation, the estimation value arithmetic circuit 212 calculates the light amount estimation value E_{λx} and the shape information k1 to km. The estimation value arithmetic circuit 212 sends the calculated light amount estimation value E_{λx} and shape information k1 to km to the evaluation value arithmetic circuit 214.

In step S203, the evaluation value arithmetic circuit 214 of the shape optimization circuit 200 acquires the light amount D_{λx} detected by the photodetector 320 from the photodetector 320 via the input circuit 130. In step S204, the evaluation value arithmetic circuit 214 of the controller 100 calculates the evaluation value J by using the equation (11), based on the light amount estimation value E_{λx} acquired from the estimation value arithmetic circuit 212 and the light amount D_{λx} acquired from the photodetector 320.

In step S205, the evaluation value arithmetic circuit 214 determines whether the evaluation value J is equal to or less than the predetermined threshold A. If it is determined that the evaluation value J is not equal to or less than the predetermined threshold A, the process returns to step S202. Specifically, the evaluation value arithmetic circuit 214 causes the estimation value arithmetic circuit 212 to calculate once again the light amount estimation value E_{λx}. On the other hand, in step S205, if it is determined that the evaluation value J is equal to or less than the predetermined threshold A, the process advances to step S206. In step S206, the evaluation value arithmetic circuit 214 of the controller 100 outputs the shape information k1 to km calculated by the estimation value arithmetic circuit 212. Thereafter, the process returns to the process described with reference to FIG. 16.

Referring back to FIG. 16, a further description will be given. In step S104, the endoscope shape calculator 140 of the controller 100 calculates the shape of the insertion section 812 of the endoscope 810, based on the shape information k1 to km calculated by the shape optimization circuit 200 and the position information of the sensing parts 410. Since the distance between the respective sensing parts 410 is included in the position information, the shape of the insertion section 812 is calculated if the positions of the respective sensing parts 410 are connected based on the shape information k1 to km. In step S105, the controller 100 causes the display 180 to display the calculated shape of the insertion section 812.

In step S106, the controller 100 determines whether an instruction to end the operation has been input. If the instruction to end the operation has not been input, the process returns to step S102 and repeats the above-described operation. On the other hand, if the instruction to end the operation has been input, the process relating to this operation is terminated. Such a configuration may be adopted that the present operation is terminated also when the instruction to end the operation has been input in the repeating operation of step S102, or that the present operation is terminated also when the instruction to end the operation has been input in the repeating operation of step S202 to step S205.

Although the example in which the optimal light amount estimation value E_{λx} is calculated by the repetitive arithmetic operations was shown, a method may be used in which the optimal light amount estimation value E_{λx} is calculated for the light amount D_{λx} acquired by a one-time arithmetic operation, such as least squares method. In the optimization arithmetic operation, use may be made of a light amount estimation table expressed by the form of a table, not limited to the light amount estimation equation expressed by the form of a function. The light amount estimation relationships, which can be expressed in such various forms, are stored in the storage circuit 120. In this manner, use may be made of various optimization arithmetic operations by which the optimal light amount estimation value E_{λx} can be calculated for the light amount D_{λx} acquired by the photodetector 320.

Additionally, the evaluation value J is not limited to equation (11), and may be any value which properly expresses the difference between the light amount D_{λx} detected by the photodetector 320 and the calculated optimal light amount estimation value E_{λx}.

According to the above operation, the shape estimation device 10 can calculate the shape of the insertion section 812 of the endoscope 810. According to the present embodiment, the sensor unit 400 is generally composed of only the light guide 420 which is, for example, an optical fiber. Furthermore, the information of each of the plural sensing parts 410 is separated by the difference in wavelength. These are advantageous in reducing the size of the insertion section 812.

According to the first calculation method, the shape of each sensing part 410 can be easily calculated with high precision by the execution of the optimization arithmetic operation. In addition, in the first calculation method, the number of wavelengths which are used, that is, p in equation (11), can be arbitrarily set if this number is set to be the number of sensing parts, i.e., m, or more. By making p greater, the shapes of sensing parts 410 can be calculated more precisely.

The shape estimation device 10 according to the embodiment can be used for estimating shapes of various kinds of articles. Specifically, the shape estimation device 10 can be used for estimating shapes of endoscopes for medical use and industrial use, can be used for estimating the shapes of, for example, a catheter and a surgery-assisting robot, and can be used for estimating the shapes of various robots and deformable instruments, not limited to medical uses.

[Second Calculation Method]

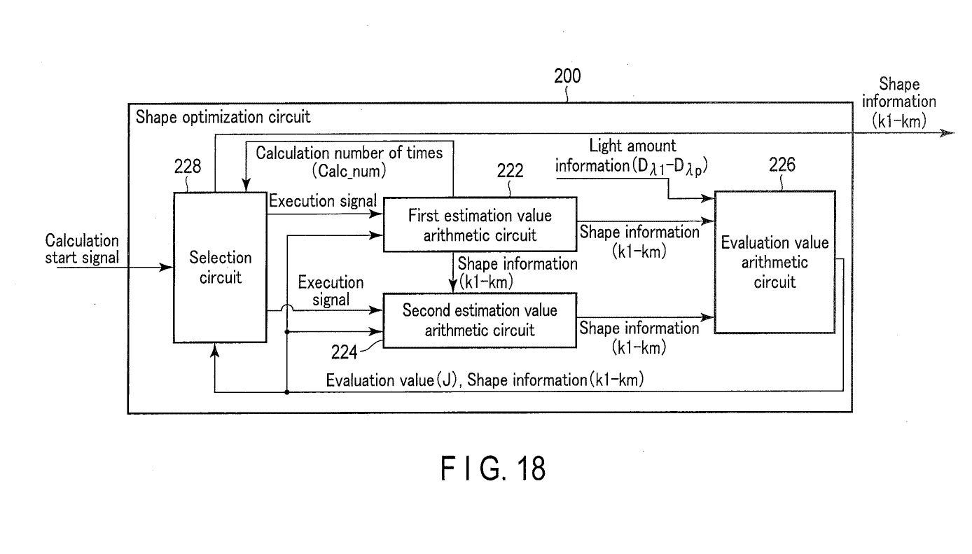

A second calculation method will be described. Different points from the first calculation method will be described, and a description of common parts is omitted. FIG. 18 shows the outline of a configurational example of a shape optimization circuit 200 according to the second calculation method. As shown in FIG. 18, the shape optimization circuit 200 includes a first estimation value arithmetic circuit 222, a second estimation value arithmetic circuit 224, an evaluation value arithmetic circuit 226, and a selection circuit 228. In this manner, the second calculation method differs from the first calculation method in that the two estimation value arithmetic circuits are included in the shape optimization circuit 200.

The first estimation value arithmetic circuit 222 executes a first optimization arithmetic operation. The first optimization arithmetic operation is a global optimization arithmetic operation. Here, the global optimization arithmetic operation is a method which can derive an optimal solution without falling into a local solution, such as Particle Swarm Optimization (PSO), Differential Evolution (DE), Genetic Algorithm (GA) and Simulated Annealing (SA). The first estimation value arithmetic circuit 222 outputs the light amount estimation value E_{λx} and shape information k1 to km, which were calculated by the global optimization arithmetic operation, to the evaluation value arithmetic circuit 226. In addition, the first estimation value arithmetic circuit 222 may output, where necessary, the shape information k1 to km to the second estimation value arithmetic circuit 224. Further, the first estimation value arithmetic circuit 222 counts the calculation number of times, and retains the counted result as a calculation number of times Calc_num. Moreover, the first estimation value arithmetic circuit 222 outputs the calculation number of times Calc_num to the selection circuit 228.

On the other hand, the second estimation value arithmetic circuit 224 executes a second optimization arithmetic operation. The second optimization arithmetic operation is a local optimization arithmetic operation with quick convergence. Here, the optimization arithmetic operation with quick convergence is a neighborhood search method for finding a local solution, such as Newton's method, a steepest descent method, and a simplex method. The second estimation value arithmetic circuit 224 outputs the light amount estimation value E_{λx} and shape information k1 to km, which were calculated by the local optimization arithmetic operation, to the evaluation value arithmetic circuit 226.

The above-described first arithmetic operation and second arithmetic operation are merely examples. The first arithmetic operation requires a longer calculation time than the second arithmetic operation, but can derive a proper solution with high precision. On the other hand, there is concern that the second arithmetic operation, compared to the first arithmetic operation, may derive, for example, an improper local solution, but the calculation time in the second arithmetic operation is shorter. If the first arithmetic operation has higher precision than the second arithmetic operation, and if the second arithmetic operation has quicker convergence than the first arithmetic operation, the first arithmetic operation and second arithmetic operation may be any combination of methods.

The evaluation value arithmetic circuit 226 calculates the evaluation value J by using the equation (11), based on the light amount estimation value E_{λx} acquired from the first estimation value arithmetic circuit 222 or second estimation value arithmetic circuit 224, and the light amount D_{λx} acquired by the photodetector 320. The evaluation value arithmetic circuit 226 outputs the evaluation value J and shape information k1 to km to the selection circuit 228.

A calculation start signal is input to the selection circuit 228. When the calculation start signal has been input, the selection circuit 228 causes the first estimation value arithmetic circuit 222 to start the optimization arithmetic operation. In addition, based on the evaluation value J and shape information k1 to km acquired from the evaluation value arithmetic circuit 226 and the calculation number of times Calc_num acquired from the first estimation value arithmetic circuit 222, the selection circuit 228 selects and executes one of an operation of causing the first estimation value arithmetic circuit 222 to execute the optimization arithmetic operation, an operation of causing the second estimation value arithmetic circuit 224 to execute the optimization arithmetic operation, and an operation of outputting the shape information k1 to km which is the result of the arithmetic operation. When the selection circuit 228 causes the first estimation value arithmetic circuit 222 to execute the optimization arithmetic operation, the selection circuit 228 outputs the currently retained shape information k1 to km to the first estimation value arithmetic circuit 222. In addition, when the selection circuit 228 causes the second estimation value arithmetic circuit 224 to execute the optimization arithmetic operation, the selection circuit 228 outputs the currently retained shape information k1 to km to the second estimation value arithmetic circuit 224.

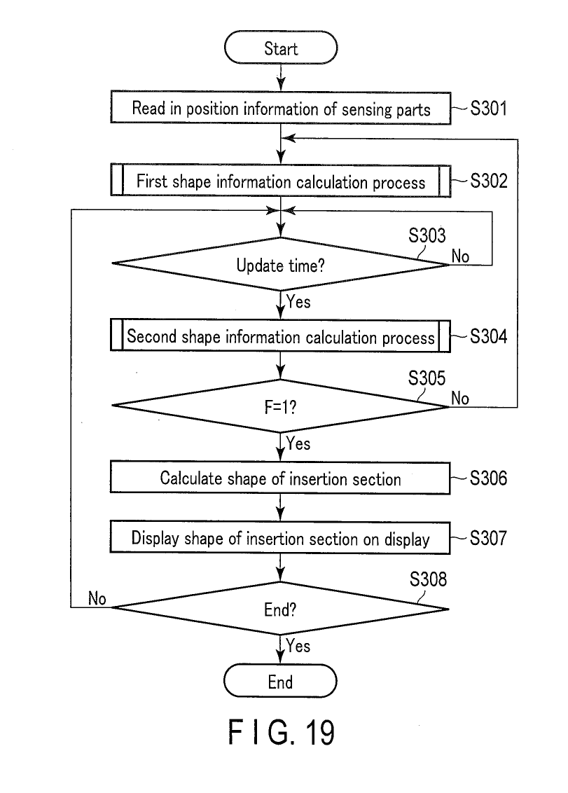

The operation of the controller 100 using the second calculation method will now be described with reference to a flowchart of FIG. 19. In step S301, the controller 100 reads in position information of the sensing parts 410 stored in the storage circuit 120.

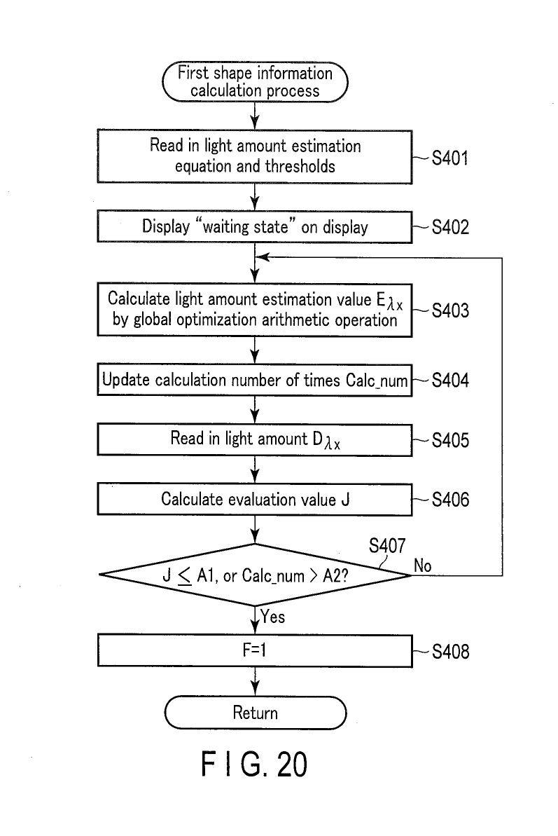

The process of step S302 to step S305 is executed by the shape optimization circuit 200. In step S302, the shape optimization circuit 200 of the controller 100 executes a first shape information calculation process. Referring to a flowchart of FIG. 20, the first shape information calculation process will be described.

In step S401, the selection circuit 228 of the shape optimization circuit 200 reads in various thresholds which are stored the storage circuit 120. In addition, the first estimation value arithmetic circuit 222 of the shape optimization circuit 200 reads in the light amount estimation equation stored in the storage circuit 120. In step S402, the shape optimization circuit 200 causes the display 180 to display, for example, “waiting state”.

In step S403, the first estimation value arithmetic circuit 222 of the shape optimization circuit 200 calculates the light amount estimation value E_{λx} by the global optimization arithmetic operation. In step S404, the first estimation value arithmetic circuit 222 of the shape optimization circuit 200 updates the calculation number of times Calc_num. Specifically, the calculation number of times Calc_num is updated as follows:

Calc_num=Calc_num+1

The first estimation value arithmetic circuit 222 outputs the light amount estimation value E_{λx} and shape information k1 to km, which are the calculation result, to the evaluation value arithmetic circuit 226. In addition, the first estimation value arithmetic circuit 222 outputs the calculation number of times Calc_num to the selection circuit 228. Besides, since the second estimation value arithmetic circuit 224 utilizes the light amount estimation value E_{λx} and shape information k1 to km, the first estimation value arithmetic circuit 222 may output the light amount estimation value E_{λx} and shape information k1 to km to the second estimation value arithmetic circuit 224. Specifically, the second estimation value arithmetic circuit 224 may acquire the light amount estimation value E_{λx} to be used in the arithmetic operation, from the evaluation value arithmetic circuit 226, or from the first estimation value arithmetic circuit 222.

In step S405, the evaluation value arithmetic circuit 226 of the shape optimization circuit 200 reads in the light amount D_{λx} from the photodetector 320. In step S406, the evaluation value arithmetic circuit 226 of the shape optimization circuit 200 calculates the evaluation value J by using the equation (11), based on the light amount estimation value E_{λx} and calculation number of times Calc_num, which were acquired from the first estimation value arithmetic circuit 222, and the light amount D_{λx} acquired from the photodetector 320. The evaluation value arithmetic circuit 226 outputs to the selection circuit 228 the evaluation value J, and the light amount estimation value E_{λx} and shape information k1 to km, which were acquired from the first estimation value arithmetic circuit 222.

In step S407, the selection circuit 228 of the shape optimization circuit 200 determines whether the evaluation value J is equal to or less than the predetermined threshold A, or whether Calc_num is greater than a predetermined threshold A2. If the evaluation value J is greater than the predetermined threshold A, and Calc_num is not greater than the predetermined threshold A2, the process returns to step S403. At this time, the calculation of the light amount estimation value E_{λx} is repeatedly executed. On the other hand, if the evaluation value J is equal to or less than the predetermined threshold A, or if Calc_num is greater than the predetermined threshold A2, the process advances to step S408.

In step S408, the selection circuit 228 of the shape optimization circuit 200 sets “1” as the value of a flag F which indicates that the global optimization arithmetic operation has properly been finished. Thereafter, the process returns to the process described with reference to FIG. 19.

Referring back to FIG. 19, a further description will be given. After the first shape information calculation process, in step S303, the selection circuit 228 included in the shape optimization circuit 200 of the controller 100 determines whether a predetermined update time has passed. If it is determined that the predetermined update time has not passed, the process repeats step S303, and stands by. On the other hand, if it is determined that the predetermined update time has passed, the process advances to step S304.

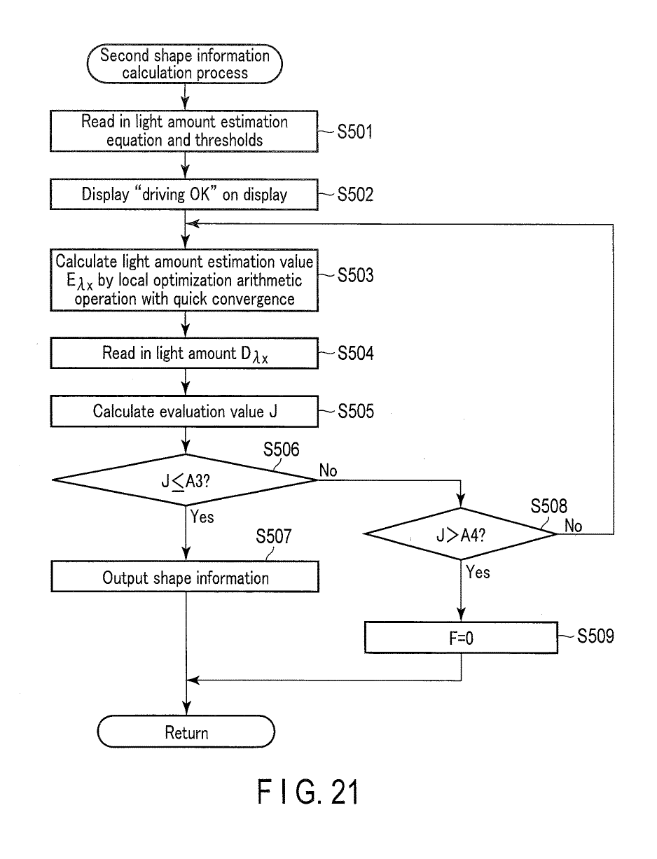

In step S304, the shape optimization circuit 200 of the controller 100 executes a second shape information calculation process. Referring to a flowchart of FIG. 21, the second shape information calculation process will be described.

In step S501, the selection circuit 228 of the shape optimization circuit 200 reads in various thresholds which are stored the storage circuit 120. In addition, the second estimation value arithmetic circuit 224 of the shape optimization circuit 200 reads in the light amount estimation equation stored in the storage circuit 120. In step S502, the selection circuit 228 of the shape optimization circuit 200 causes the display 180 to display, for example, “driving OK”.

In step S503, the second estimation value arithmetic circuit 224 of the shape optimization circuit 200 calculates the light amount estimation value E_{λx} by the local optimization arithmetic operation with quick convergence. The second estimation value arithmetic circuit 224 outputs the light amount estimation value E_{λx} and shape information k1 to km, which are the calculation result, to the evaluation value arithmetic circuit 226.

In step S504, the evaluation value arithmetic circuit 226 of the shape optimization circuit 200 reads in the light amount D_{λx} from the photodetector 320. In step S505, the evaluation value arithmetic circuit 226 of the shape optimization circuit 200 calculates the evaluation value J by using the equation (11), based on the light amount estimation value E_{λx} acquired from the second estimation value arithmetic circuit 224, and the light amount D_{λx} acquired from the photodetector 320. The evaluation value arithmetic circuit 226 outputs to the selection circuit 228 the evaluation value J, and the light amount estimation value E_{λx} and shape information k1 to km, which were acquired from the second estimation value arithmetic circuit 224.

In step S506, the selection circuit 228 of the shape optimization circuit 200 determines whether the evaluation value J is equal to or less than a predetermined threshold A3. Here, the threshold A3 is less than the threshold A1. If the evaluation value J is equal to or less than the predetermined threshold A3, the process advances to step S507. In step S507, the selection circuit 228 of the shape optimization circuit 200 outputs the shape information k1 to km of the sensing parts calculated by the light amount estimation equation, and the calculation number of times Calc_num, to, for example, the endoscope shape calculator 140 or output circuit 160. Thereafter, the process returns to the process described with reference to FIG. 19.

In the determination of step S506, if it is determined that the evaluation value J is not equal to or less than the predetermined threshold A3, the process goes to step S508. In step S508, the selection circuit 228 of the shape optimization circuit 200 determines whether the evaluation value J is greater than a predetermined threshold A4. Here, the predetermined threshold A4 is greater than the predetermined threshold A1. If it is determined that the evaluation value J is not greater than the predetermined threshold A4, the process returns to step S503. At this time, the calculation of the light amount estimation value E_{λx} is repeatedly executed. On the other hand, if it is determined that the evaluation value J is greater than the predetermined threshold A4, the process advances to step S509. In step S509, the selection circuit 228 of the shape optimization circuit 200 sets “0” as the value of the flag F. Thereafter, the process returns to the process described with reference to FIG. 19.

Referring back to FIG. 19, a further description will be given. After the second shape information calculation process, in step S305, the selection circuit 228 included in the shape optimization circuit 200 of the controller 100 determines whether the flag F is “1” or not. If the flag F is not “1”, the process returns to step S302. Specifically, while the second shape information calculation process is being executed, if convergence to an improper solution is about to occur, the flag F becomes “0” in step S509, and the first shape information calculation process is executed once again. On the other hand, if it is determined that the flag F is “1”, the process advances to step S306. In this manner, the convergence to an improper solution by the second shape information calculation process with relatively low precision can be prevented.

In step S306, the endoscope shape calculator 140 of the controller 100 calculates the shape of the insertion section 812 of the endoscope 810, based on the shape information k1 to km calculated by the shape optimization circuit 200 and the position information of the sensing parts 410. In step S307, the controller 100 causes the display 180 to display the calculated shape of the insertion section 812. In step S308, the controller 100 determines whether an instruction to end the operation has been input. If the instruction to end the operation has not been input, the process returns to step S303. On the other hand, if the instruction to end the operation has been input, the process relating to this operation is terminated.

According to the process using the second calculation method, while the calculation of an improper local solution is prevented by the global optimization arithmetic operation by the first estimation value arithmetic circuit 222, proper shape information can be quickly calculated by the optimization arithmetic operation with high convergence by the second estimation value arithmetic circuit 224. According to the second calculation method, the calculation of shape information, which is quick and exact as a whole, can be realized.

While the global optimization arithmetic operation by the first estimation value arithmetic circuit 222 is being executed, it is preferable that the shape of the insertion section 812 does not vary, in order to converge the arithmetic operation. Thus, “waiting state” is displayed on the display 180, and the user is prompted not to move the insertion section 812. In addition, while the optimization arithmetic operation with high convergence by the second estimation value arithmetic circuit 224 is being executed, it is possible to adapt to a change in shape of the insertion section 812, and thus “driving OK” is displayed on the display 180. By such display, the arithmetic operation is prevented from failing to converge. The display of “waiting state” or “driving OK” is merely an example, and any kind of display may be adopted. In addition, not limited to display, such a configuration as to notify the user by sound may be adopted.

In the second calculation method, the selection circuit 228 selects the execution of the first optimization arithmetic operation or the execution of the second optimization arithmetic operation. However, the shape estimation device 10 may be configured to enable the user to make such selection. In this case, the shape estimation device 10 is provided with a selection input circuit for the user to input his/her own selection. By the configuration which enables the user to select the kind of arithmetic operation, the user can select by himself/herself either the exactness or the quickness of display, which is necessary for the user.

[Third Calculation Method]

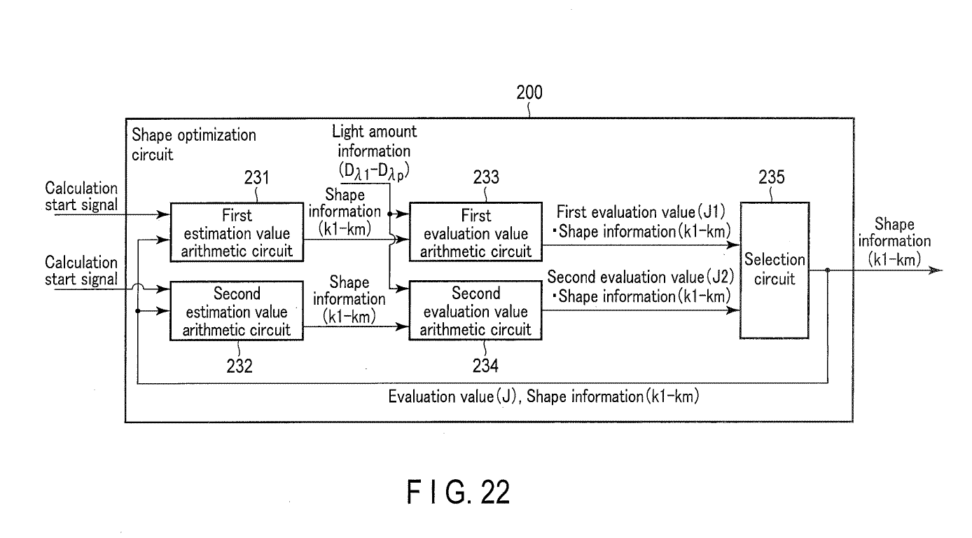

A third calculation method will be described. Different points from the second calculation method will be described, and a description of common parts is omitted. FIG. 22 shows the outline of a configurational example of a shape optimization circuit 200 according to the third calculation method. As shown in FIG. 22, the shape optimization circuit 200 includes a first estimation value arithmetic circuit 231, a second estimation value arithmetic circuit 232, a first evaluation value arithmetic circuit 233, a second evaluation value arithmetic circuit 234, and a selection circuit 235.

Like the first estimation value arithmetic circuit 222 in the second calculation method, the first estimation value arithmetic circuit 231 is an estimation value arithmetic circuit which executes the global optimization arithmetic operation. The first estimation value arithmetic circuit 231 calculates a first light amount estimation value E_{λx1} based on first shape information k1′ to km′. The first estimation value arithmetic circuit 231 outputs the first light amount estimation value E_{λx1} and first shape information k1′ to km′ to the first evaluation value arithmetic circuit 233.

The first evaluation value arithmetic circuit 233 calculates, based on the following equation (12), a first evaluation value J1 for the first light amount estimation value E_{λx1} calculated by the first estimation value arithmetic circuit 231.

$$J 1 = {\sum }_{x = 1}^{p} {( {D}_{λ x} - {E}_{λ x 1} ( k {1}^{′} , … , {km}^{′} ) )}^{2} \qquad equation ( 12 )$$

The first evaluation value arithmetic circuit 233 outputs the first evaluation value J1 and first shape information k1′ to km′ to the selection circuit 235.

Like the second estimation value arithmetic circuit 224 in the second calculation method, the second estimation value arithmetic circuit 232 is an estimation value arithmetic circuit which executes the local optimization arithmetic operation. The second estimation value arithmetic circuit 232 calculates a second light amount estimation value E_{λx2} based on second shape information k1″ to km″. The second estimation value arithmetic circuit 232 outputs the second light amount estimation value E_{λx2} and second shape information k1″ to km″ to the second evaluation value arithmetic circuit 234.

The second evaluation value arithmetic circuit 234 calculates, based on the following equation (13), a second evaluation value J2 for the second light amount estimation value E_{λx2} calculated by the second estimation value arithmetic circuit 232.

$$J 2 = {\sum }_{x = 1}^{p} {( {D}_{λ x} - {E}_{λ x 1} ( k {1}^{″} , … , {km}^{″} ) )}^{2} \qquad equation ( 13 )$$

The second evaluation value arithmetic circuit 234 outputs the second evaluation value J2 and second shape information k1″ to km″ to the selection circuit 235.

In the third calculation method, the global optimization arithmetic operation by the first estimation value arithmetic circuit 231 and first evaluation value arithmetic circuit 233 and the local optimization arithmetic operation by the second estimation value arithmetic circuit 232 and second evaluation value arithmetic circuit 234 are executed in parallel.

The selection circuit 235 determines the evaluation value J, based on the first evaluation value J1 and second evaluation value J2. Based on the evaluation vale J, the selection circuit 235 determines whether the optimization arithmetic operation is repeatedly executed, or the arithmetic operation is finished and the calculation result is output. The selection circuit 235 outputs, as shape information k1 to km, either the first shape information k1′ to km′ or the second shape information k1″ to km″, which is more appropriate.

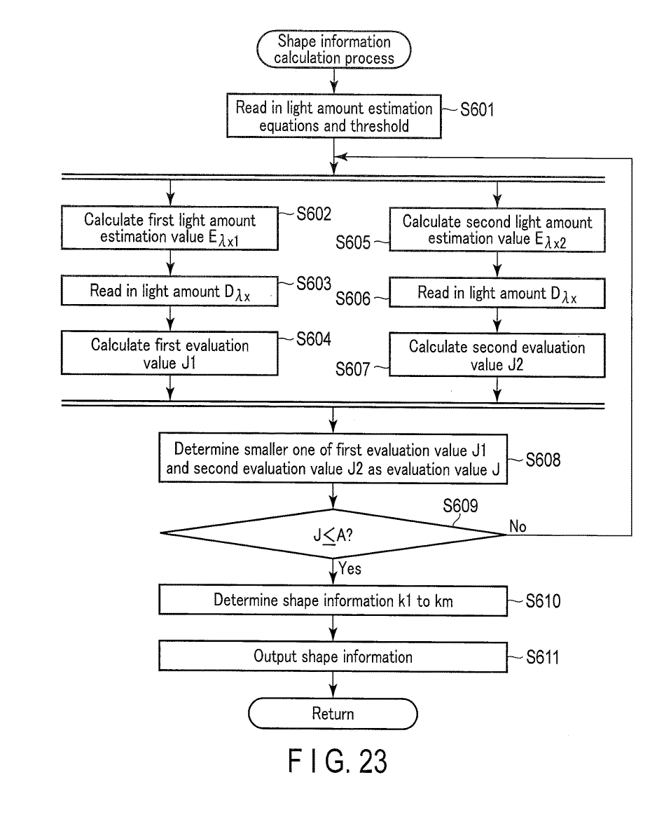

The operation of the controller 100 using the third calculation method will be described. This operation is similar to the operation of the controller 100 using the first calculation method described with reference to FIG. 16. However, the operation of the shape information calculation process is different. Referring to a flowchart of FIG. 23, a description will be given of the operation of the shape information calculation process using the third calculation method.

In step S601, the shape optimization circuit 200 reads in the light amount estimation equation and threshold. Specifically, the first estimation value arithmetic circuit 231 reads in the light amount estimation equation to be used in the calculation of the first light amount estimation value E_{λx1}. The second estimation value arithmetic circuit 232 reads in the light amount estimation equation to be used in the calculation of the second light amount estimation value E_{λx2}. The selection circuit 235 reads in the threshold A.

The process of step S602 to step S604 by the first estimation value arithmetic circuit 231 and first evaluation value arithmetic circuit 233 and the process of step S605 to step S607 by the second estimation value arithmetic circuit 232 and second evaluation value arithmetic circuit 234 to be executed after step S601, are parallel processes.

In step S602, the first estimation value arithmetic circuit 231 of the shape optimization circuit 200 calculates the first light amount estimation value E_{λx1}. The first estimation value arithmetic circuit 231 outputs the calculated first light amount estimation value E_{λx1} to the first evaluation value arithmetic circuit 233.

In step S603, the first evaluation value arithmetic circuit 233 of the shape optimization circuit 200 reads in the light amount D_{λx} from the photodetector 320. In step S604, the first evaluation value arithmetic circuit 233 of the shape optimization circuit 200 calculates the first evaluation value J1 by using the equation (12), based on the first light amount estimation value E_{λx1} acquired from the first estimation value arithmetic circuit 231 and the light amount D_{λx} acquired from the photodetector 320. The first evaluation value arithmetic circuit 233 outputs to the selection circuit 235 the calculated first evaluation value J1 and first shape information k1′ to km′ relating to the first light amount estimation value E_{λx1}.

In step S605, the second estimation value arithmetic circuit 232 of the shape optimization circuit 200 calculates the second light amount estimation value E_{λx2}. The second estimation value arithmetic circuit 232 outputs the calculated second light amount estimation value E_{λx2} to the second evaluation value arithmetic circuit 234.

In step S606, the second evaluation value arithmetic circuit 234 of the shape optimization circuit 200 reads in the light amount D_{λx} from the photodetector 320. In step S607, the second evaluation value arithmetic circuit 234 of the shape optimization circuit 200 calculates the second evaluation value J2 by using the equation (13), based on the second light amount estimation value E_{λx2} acquired from the second estimation value arithmetic circuit 232 and the light amount D_{λx} acquired from the photodetector 320. The second evaluation value arithmetic circuit 234 outputs to the selection circuit 235 the calculated second evaluation value J2 and second shape information k1″ to km″ relating to the second light amount estimation value E_{λx2}.

After the process of step S602 to step S604 and the process of step S605 to step S607, the selection circuit 235 of the shape optimization circuit 200 compares, in step S608, the first evaluation value J1 acquired from the first evaluation value arithmetic circuit 233 and the second evaluation value J2 acquired from the second evaluation value arithmetic circuit 234, and determines the smaller one as the evaluation value J.

In step S609, the selection circuit 235 of the shape optimization circuit 200 determines whether the evaluation value J is equal to or less than the predetermined threshold A. If it is determined that the evaluation value J is not equal to or less than the predetermined threshold A, the process returns to the parallel arithmetic operations of step S602 and step S605. At this time, the first estimation value arithmetic circuit 231 and second estimation value arithmetic circuit 232 may use the shape information calculated by themselves in the previous arithmetic operations, or the first estimation value arithmetic circuit 231 and second estimation value arithmetic circuit 232 may both use the shape information of the smaller evaluation value. On the other hand, if it is determined that the evaluation value J is equal to or less than the predetermined threshold A, the process advances to step S610.

In step S610, the selection circuit 235 of the shape optimization circuit 200 compares the first evaluation value J1 and second evaluation value J2. When the first evaluation value J1 is smaller, the selection circuit 235 determines the first shape information k1″ to km″ as the shape information k1 to km. In addition, when the second evaluation value J2 is smaller, the selection circuit 235 determines the second shape information k1″ to km″ as the shape information k1 to km.

In step S611, the selection circuit 235 of the shape optimization circuit 200 outputs the shape information k1 to km determined in step S610, to, for example, the endoscope shape calculator 140. Thereafter, the process returns to the process described with reference to FIG. 16.

If the third calculation method is used, a proper optimal solution can be quickly calculated by the parallel arithmetic operations of the global optimization arithmetic operation by the first estimation value arithmetic circuit 231 and the optimization arithmetic operation with high convergence by the second estimation value arithmetic circuit 232. Furthermore, while the global optimization arithmetic operation does not properly converge, the result of the optimization arithmetic operation with high convergence is output. When the global optimization arithmetic operation properly converges, the result of the global optimization arithmetic operation with high precision is output. Therefore, unlike the case of the second calculation method, the shape information can constantly be output.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A shape estimation device comprising:
an input circuit configured to receive light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts;
a storage circuit configured to store a light amount estimation relationship including shape characteristic information representing a relationship between the shape, the wavelength and the light amount with respect to each of the plurality of sensing parts; and
a shape arithmetic circuit configured to calculate shape information being an estimation value of the shape by calculating a light amount estimation value by an optimization arithmetic operation such that the light amount estimation value calculated based on the light amount estimation relationship and the light amount information satisfy a predetermined condition, the light amount estimation value which is a relationship between the wavelength and the light amount.

2. The shape estimation device according to claim 1, wherein the optimization arithmetic operation to be executed by the shape arithmetic circuit includes at least two kinds of arithmetic methods,
at least one of the at least two kinds of arithmetic methods includes a convergence calculation, and
the at least two kinds of arithmetic methods have different convergences.

3. The shape estimation device according to claim 2, wherein the at least two kinds of arithmetic methods include a first optimization arithmetic operation and a second optimization arithmetic operation,
the first optimization arithmetic operation has a higher precision than the second optimization arithmetic operation, and
the second optimization arithmetic operation has a higher convergence than the first optimization arithmetic operation.

4. The shape estimation device according to claim 3, wherein the shape arithmetic circuit is configured to successively execute the at least two kinds of arithmetic methods.

5. The shape estimation device according to claim 4, wherein the shape arithmetic circuit is configured to execute the second optimization arithmetic operation after executing the first optimization arithmetic operation.

6. The shape estimation device according to claim 5, wherein the shape arithmetic circuit is configured to use, in the second optimization arithmetic operation, the shape information calculated by the first optimization arithmetic operation.

7. The shape estimation device according to claim 3, wherein the shape arithmetic circuit is configured:
to execute the second optimization arithmetic operation after executing the first optimization arithmetic operation,
to use, in the second optimization arithmetic operation, the shape information calculated by the first optimization arithmetic operation, and
to suspend the second optimization arithmetic operation and execute the first optimization arithmetic operation, when an error between the light amount information and the light amount estimation value has become greater than a predetermined threshold while the shape arithmetic circuit is executing the second optimization arithmetic operation.

8. The shape estimation device according to claim 2, wherein the shape arithmetic circuit is configured:
to execute, in parallel, operations using the at least two kinds of arithmetic methods, respectively, and
to select optimal said shape information from between at least two pieces of the shape information obtained as results of the arithmetic operations.

9. The shape estimation device according to claim 8, wherein the shape arithmetic circuit is configured to select the shape information, based on a magnitude of an error between the light amount information and the light amount estimation value.

10. The shape estimation device according to claim 1, wherein the optimization arithmetic operation includes a convergence calculation, and
the shape arithmetic circuit is configured to terminate the convergence calculation and determine the shape information, when an error between the light amount information and the light amount estimation value has become less than a predetermined threshold.

11. The shape estimation device according to claim 3, further comprising a selection input circuit configured to receive an input of selection between the first optimization arithmetic operation and the second optimization arithmetic operation,
wherein the shape arithmetic circuit is configured to execute either the first optimization arithmetic operation or the second optimization arithmetic operation, in accordance with the input to the selection input circuit.

12. The shape estimation device according to claim 3, wherein the shape arithmetic circuit is configured to output a signal indicative of a waiting state, while the shape arithmetic circuit is executing the first optimization arithmetic operation.

13. The shape estimation device according to claim 12, wherein the signal indicative of the waiting state is a signal for causing a display to display the wait state.

14. The shape estimation device according to claim 1, wherein the shape arithmetic circuit is configured to store a result of the optimization arithmetic operation previously executed.

15. The shape estimation device according to claim 1, further comprising:
a light source configured to emit light; and
the sensor,
the sensor including:
a light guide configured to guide the light emitted from the light source;
a plurality of the sensing parts including a plurality of optical members provided on the light guide and configured to exert mutually different effects on a spectrum of light guided by the light guide; and
a photodetector configured to detect light guided by the light guide and affected by the plurality of optical members, and to output the light amount information.

16. An endoscope system comprising:
the shape estimation device according to claim 15;
an endoscope configured such that the light guide is provided in an insertion section; and
an endoscope shape calculator configured to calculate a shape of the insertion section, based on the shape information.

17. A shape estimation method comprising:
acquiring light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts;
acquiring a light amount estimation relationship including shape characteristic information representing a relationship between the shape, the wavelength and the light amount with respect to each of the plurality of sensing parts; and
calculating shape information being an estimation value of the shape, by calculating a light amount estimation value by an optimization arithmetic operation such that the light amount estimation value calculated based on the light amount estimation relationship and the light amount satisfy a predetermined condition, the light amount estimation value which is a relationship between the wavelength and the light amount.

18. A program for shape estimation, which causes a computer to execute:
acquiring light amount information which is a relationship between a wavelength and a light amount, the light amount information being acquired by using a sensor configured such that the light amount to be detected with respect to the wavelength corresponding to each of a plurality of sensing parts varies in accordance with a shape of each of the plurality of sensing parts;
acquiring a light amount estimation relationship including shape characteristic information representing a relationship between the shape, the wavelength and the light amount with respect to each of the plurality of sensing parts; and
calculating shape information being an estimation value of the shape, by calculating a light amount estimation value by an optimization arithmetic operation such that the light amount estimation value calculated based on the light amount estimation relationship and the light amount information satisfy a predetermined condition, the light amount estimation value which is a relationship between the wavelength and the light amount.
